(19) United States
(12) Patent Application Publication
(10) Pub. No.: US 2025/0295824 A1
(43) Pub. Date: Sep. 25, 2025

(54) ULTRAVIOLET LIGHT SOURCE WITH ACOUSTIC FLOW SENSOR

(71) Applicant: TROJAN TECHNOLOGIES GROUP ULC, London (CA)

(72) Inventors: Rahma SYED, London (CA); Jeremy SILVER-MAHR, London (CA)

(73) Assignee: TROJAN TECHNOLOGIES GROUP ULC, London (CA)

(21) Appl. No.: 19/082,886

(22) Filed: Mar. 18, 2025

Related U.S. Application Data
(60) Provisional application No. 63/567,206, filed on Mar. 19, 2024.

(51) Int. Cl. A61L 2/10; G01F 1/66

(52) U.S. Cl. CPC A61L 2/10; G01F 1/662; A61L 2202/11; A61L 2202/14

(57) ABSTRACT

An apparatus includes a reactor, a UV light source assembly, a vibration detection sensor, and controller, the controller being configured to receive a signal from the vibration detection sensor based on vibrations detected by the vibration detection sensor and control an intensity of UV light produced by the UV light source assembly based on a value of the signal. The apparatus may further include an acoustic signal generator and in an embodiment, the acoustic generator may be a pair of discs positioned in an inlet of the reactor or a bent portion of the inlet. In another embodiment, the vibrations detected by the vibration detection sensor may be vibrations generated by a device other than the apparatus, such as a faucet or a valve.

### TECHNICAL FIELD

This application relates to systems and methods for treating fluids with ultraviolet light.

### BACKGROUND

Proper disinfection of water is critical to ensure water quality. As the need for cleaner sources of water has increased, water disinfection methods have evolved to match the rising challenge. Water sources may contain heavy metals, sediment, chemicals, pesticides, or the like. Water sources may also contain pathogens such as microorganisms, viruses, or the like. Left untreated, such water may be unhealthy or unsafe for use by humans or animals. Ultraviolet light treatment of water may be used to inactivate pathogens. Water may pass through a treatment chamber where the water is subjected to ultraviolet light. The ultraviolet treatment may damage nucleic acids of the pathogens making the pathogens incapable of performing vital cellular functions, thereby rendering them harmless. Thus, this ultraviolet treatment process may make water potable despite the water source containing microorganisms, viruses, or the like.

If an ultraviolet treatment system is unable to detect when fluid is flowing, the system must be at full power at all times, thereby causing the following disadvantages. First, energy that is used to power a lamp that is used to produce the ultraviolet light is wasted when fluid is not flowing through the system. Second, there is an increase in fouling on the ultraviolet lamp that is used to produce the ultraviolet light. Third, when fluid is not flowing, water remaining in the treatment chamber will continue to increase in temperature, thereby causing the water to be delivered to the customer at a higher temperature than intended. One solution to these problems is to modulate an amount of power sent to produce the ultraviolet light based on the amount of flow traveling through the chamber. A flow sensor may be used to detect the flow rate of the fluid traveling through the reactor vessel. Other fluid treatment systems have used stand-alone flow sensors to detect the flow rate of the fluid traveling through the reactor vessel.

The use of stand-alone flow sensors requires adding components to the fluid system that must be mounted to the system and wired to at least the lamp and a controller of the system. This adds complexity and size to the system and is particularly undesirable in residential systems, which are relatively small. As a result, the high cost and larger construction of using a stand-alone flow sensor means that residential fluid treatment systems do not generally include flow sensors.

Thus, there is a continued need for systems and methods that can detect fluid flow in the reactor in order to control the amount of ultraviolet light produced by the lamp.

### SUMMARY

In some aspects, the disclosed apparatus and methods address these issues by providing a simple and reliable acoustic flow sensor that is embedded in the reactor.

According to one aspect, this disclosure provides an apparatus for treating a fluid with ultraviolet (UV) radiation. The apparatus includes a reactor through which the fluid flows in a flow path, a UV light source that is located in the reactor and is configured to emit UV light into the fluid to treat the fluid as it flows through the reactor, a sound detection sensor that is located in the reactor and coupled to a housing of the reactor, the sound detection sensor being configured to detect sound that varies based on a flow rate of the fluid through the reactor, and to output a signal based on the detected sound, and a controller that is configured to receive the signal from the sound detection sensor and control an intensity of the UV light based on the signal.

### BRIEF DESCRIPTION OF THE DRAWINGS

FIG. 1 is a perspective view of a fluid treatment system.

FIG. 2 is a cross-sectional view of the fluid treatment system.

FIG. 3 is an exploded perspective view of the fluid treatment system showing the reactor and the controller.

FIG. 4 is a cross-sectional view of a portion of the fluid treatment system.

FIG. 5 is a partial cross-sectional view of a reactor in an alternative embodiment.

FIG. 6 is a perspective view of a circuit board that includes the flow sensor.

FIG. 7 is a schematic diagram illustrating the processing of the signal generated by the microphone.

FIG. 8 is a graph showing output voltage of the circuit board as a function of the flow rate through the fluid treatment system

### DETAILED DESCRIPTION

In the following description, numerous details are set forth to provide an understanding of the present disclosure. However, it may be understood by those skilled in the art that the systems and methods of the present disclosure may be practiced without these details and that numerous variations or modifications from the described embodiments may be possible.

Disclosed herein are fluid treatment systems, apparatuses, and methods for treating a fluid that flows through a reactor by exposing the fluid to UV light emitted from a UV light source assembly.

### Fluid Treatment System

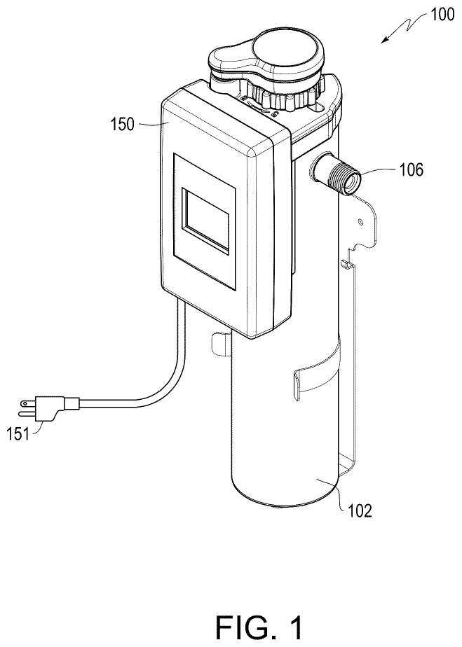

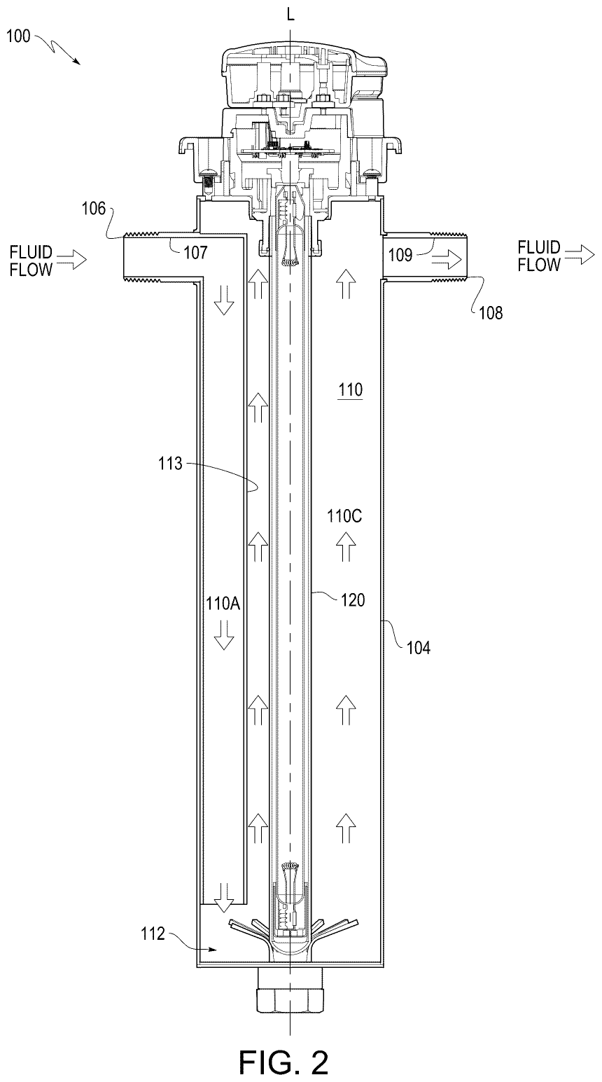

FIG. 1 is a perspective top view of an exemplary fluid treatment system 100, and FIG. 2 is a cross-sectional view of the fluid treatment system. As shown in FIGS. 1 and 2, the treatment system 100 includes a reactor 102 including a treatment chamber 110 for receiving a flow of fluid for UV radiation treatment. The treatment of the fluid with UV radiation in the treatment chamber 110 is described in greater detail below. The treatment chamber 110 extends along a longitudinal axis L and includes an inlet 106 through which fluid is introduced into the treatment chamber 110 and an outlet 108 through which the fluid is discharged from the chamber 110 after being treated. The longitudinal axis L may substantially coincide with a longitudinal axis of the reactor 102. The inlet 106 conveys the fluid to an inlet conduit 107 that extends in a direction that is transverse to the longitudinal axis L, and an outlet conduit 109 conveys the fluid to the outlet 108 where fluid is discharged from the reactor 102, e.g., into other plumbing or piping. The inlet conduit 107 and the outlet conduit 109 can extend in the same direction, and can be colinear (i.e., having the same axis or having parallel axes).

Referring to FIG. 2, the fluid flows into the apparatus at inlet 106 and is conveyed through the inlet conduit 107 in a direction perpendicular to the longitudinal axis L. The fluid then may flow along a path 110A through a tubular conduit 113 of the treatment chamber 110 in a direction parallel to the longitudinal axis L toward a bottom region 112 of the reactor 102. The fluid may then exit the tubular conduit 113 in an end region 112 of the treatment chamber 110 and reverse course by traveling along a path 110C of the treatment chamber 110 in the direction parallel to the longitudinal axis L toward a top of the treatment system 100. The fluid can surround light source assembly 120 when traveling along path 110C so that the fluid can be treated by being exposed to UV light emitted radially from the light source assembly 120, as discussed in further detail below. The inlet 106 and the outlet 108 may be arranged on opposite sides of the treatment chamber 110, perpendicular to the longitudinal axis L. However, the present disclosure is not limited to this arrangement, and the flow path through the reactor can be arranged in any suitable manner that will provide for sufficient fluid flow and UV treatment in the reactor 102.

Referring to FIG. 1, the fluid treatment system 100 may further include a controller 150 that is connected to a power source, such as an electrical grid, via a plug 151. As discussed in more detail below, the controller 150 includes at least one processor that can be configured to control the functioning and/or operation of the treatment system 100 and/or evaluate a condition of the fluid treatment system 100 and/or components thereof, including determining the flow rate of the fluid flowing through the reactor, determining whether fluid is flowing through the reactor, and/or determining whether one or more components are defective, based on measurements received from one or more sensors including, for example, one or more sound detection sensors 114, as discussed further below.

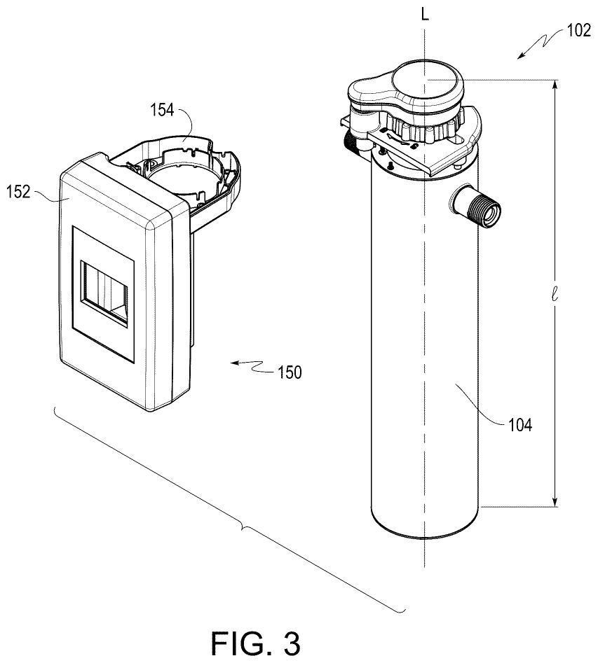

Referring to FIG. 3, the reactor 102 may be a vessel having a substantially cylindrical body defined by an outer wall 104. For example, the reactor 102 may have a circular cross-sectional shape, as shown in FIG. 3. However, the present disclosure is not limited to any particular cross-sectional shape, and the reactor 102 may have various other cross-sectional shapes, for example, an elliptical shape, a polygonal shape including, for example, a square or rectangular shape, and a semicircular shape. For residential systems, the reactor 102 may have a length l, in a direction along the longitudinal axis L from the inlet 106 to the outlet 108, in a range of 100 mm to 1,000 mm, 200 mm to 500 mm, or 240 mm to 350 mm. The treatment chamber 110 of the reactor 102 may have a diameter or width dimension in a direction orthogonal to the longitudinal axis L in a range of 25 mm to 250 mm, 50 mm to 200 mm, or 75 mm to 150 mm. The controller 150 can include a housing 152 that houses the at least one processor, and a mounting bracket 154 that fits around the reactor 102 and mounts the controller 150 to the reactor 102.

In one embodiment, the fluid treatment system 100 may be a residential system for disinfecting water for household use. The fluid treatment system 100 may be installed between a water source, such as a well or municipal water facility, and the household end use (e.g., faucet). For example, the system 100 may be installed at a point of entry of the water into the household. The system 100 can be integrated into existing piping for treating the fluid flowing through the piping. For example, the inlet 106 and the outlet 108 may be coupled to the piping to provide in-line flow and a simple connection to the piping without using an L-shape or elbow pipe connector. The system 100 may be installed so as to be integrated with the household piping in the basement of a home at a position where the water flowing from external piping in fluid communication with a well or water treatment facility enters the home. The inlet 106 may receive water flowing from the water source, the treatment chamber 110 may treat the water with UV radiation, making the water safe for use, and the outlet 108 may deliver the treated water to downstream household piping for household use, in particular for the kitchen or bathroom. For residential systems, the treatment chamber 110 can have a volume that is in a range of about 0.25 L to 10 L, from 0.5 L to 5 L, or from 1 L to 3 L, for example. The reactor 102 may be designed for a flow of fluid, such as water or other aqueous fluids, through the treatment chamber 110 at a flow rate in a range of 1 to 25 gallons per minute (gpm), 5 to 20 gpm, or 10 to 15 gpm. Of course, at times, the fluid in the reactor 102 may be substantially stagnant, in which case the flow rate may be less than 1 gpm, less than 0.5 gpm, or less than 0.25 gpm.

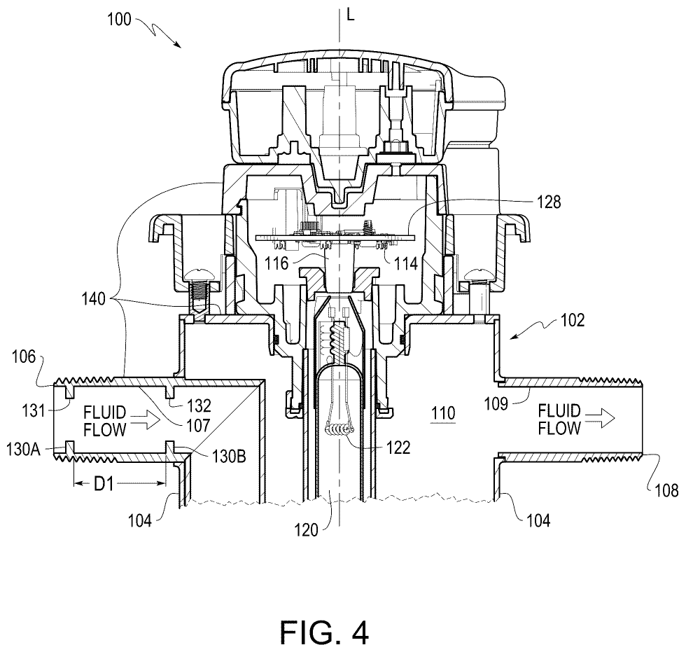

The reactor 102 may include an acoustic signal generator (i.e., a vibration generating device) that is located in the reactor and generates sound that varies based on a flow rate of the fluid. As used in this disclosure, “sound” may refer to acoustic sound waves or mechanical vibrations. As shown in FIG. 4, acoustic signal generators 130A, 130B may be positioned in the inlet conduit 107, and are used to detect when fluid is flowing through the treatment system 100. In other embodiments, one or more acoustic signal generators may be positioned in the reactor at any other suitable location along the flow path through which the fluid travels, e.g., in the outlet conduit 109, or in both the inlet conduit 107 and outlet conduit 109.

The acoustic signal generators may include at least a first disc 130A having an aperture 131 with a circular cross-section through which the fluid flows. The aperture 131 can alternatively have other cross-sectional shapes, such as a polygonal shape. In this embodiment, the first disc 130A is arranged inside the inlet conduit 107 and is configured to change a characteristic of the flow of fluid traveling through the inlet conduit 107. Specifically, the first disc 130A is used to disturb the flow of fluid thereby increasing vibration in the reactor 102 (e.g., increasing vibrations of reactor housing 140). In one aspect, the first disc 130A can increase the flow of fluid locally to generate turbulent flow in inlet conduit 107 or to increase the degree of turbulence of the fluid flow. Referring to FIG. 4, the first disc 130A is oriented in the inlet conduit 107 such that a plane extending in the radial direction of disc 130A is parallel to the longitudinal axis L and perpendicular to the direction of fluid flowing through the inlet conduit 107. However, the present disclosure is not limited to this orientation and the orientation of the radial plane of the first disc 130A may extend in any direction that intersects the walls of the inlet conduit 107 in a way that disturbs the flow of fluid. For example, the orientation of the radial plane of the first disc 130A may extend offset from the longitudinal axis L.

The aperture 131 can have a longest cross-sectional dimension (e.g., diameter in this case) that is smaller than the longest cross-sectional dimension of the inlet conduit 107. For example, the aperture 131 can have a diameter that is less than 95%, less than 75%, such as from 40% to 90% of the diameter of the inlet conduit 107. In an embodiment, the diameter of aperture 131 can be in a range of from 0.2 inches to 0.8 inches, 0.3 inches to 0.6 inches, or 0.4 inches to 0.5 inches, for example. The disc 130A can have a thickness that is in a range of 0.05 inches and 0.15 inches, 0.075 inches and 0.125 inches, and 0.09 inches and 0.11 inches. However, the diameter of the aperture 131 and thickness of disc 130A may be any suitable size for disturbing the flow of fluid within the inlet conduit 107.

In order for fluid to flow through the system at a constant rate, fluid flowing through the inlet conduit 107 at the position of the first disc 130A, i.e., through the aperture 131, must travel faster than fluid flowing through the inner diameter of the inlet 106 or other portions where the inner diameter is larger. Thus, in this arrangement, fluid flowing through the inlet conduit 107 will have different velocities along a direction of the fluid flow through the inlet conduit 107, thereby creating more turbulent regions located at positions where the slower and faster flows meet. These turbulent regions within the inlet conduit 107 can cause vibrations in the reactor housing 140, which in turn generate sound. Because the microphone 114 is coupled to the reactor housing 140, the microphone 114 can accurately detect sound generated by the acoustic signal generators. The detected sound can be used to determine whether or not fluid is flowing through the reactor 102, or to detect the flow rate of the fluid through reactor 102, without the use of a standalone flow sensor, as discussed in more detail below.

In some embodiments, and as illustrated in FIG. 4, the reactor 102 can include a second acoustic signal generator 130B that is a disc with aperture 132 in the inlet conduit 107, and is the same as the first disc 130A except that the second disc 130B is positioned downstream of the first disc 130A relative to the flow of fluid through the inlet conduit 107. The distance D1 between the first disc 130A and the second disc 130B may be in a range of 0.2 to 3 inches, 0.7 to 1.5 inches, or from 1.0 to 1.3 inches, for example. In some aspects, the distance D1 can be from 25% to 200%, from 50% to 150%, or from 100% to 130% of the diameter of the inner conduit 107. However, the distance between the first disc 130A and the second disc 130B may be any suitable amount for disturbing the flow of fluid in the inlet conduit 107 in a way that generates sufficient sound in the reactor housing 140 that can be detected by microphone 114. By including the second disc 130B, as compared to only including the first disc 130A, the fluid flowing through the inlet conduit 107 will include more positions where the slower and faster moving flows meet because the second disc 130B creates a second position where the fluid must flow through a constricted aperture 132. As a result, the combination of the two regions of faster flowing fluid through the inner diameters of the first and second discs 130A, 130B causes an increase in the amount of turbulent flow through the inlet conduit 107, thereby generating an increased amount of sound that can be detected.

The first and second discs 130A, 130B may be machined as a single component together with the inlet conduit 107 such that the outer diameters of the discs 130A, 130B are integral with an inner diameter of the inlet conduit 107. Alternatively, the acoustic sound generators may be separate components that are attached in the reactor housing 140 via press fitting, welding, or any other suitable means. The acoustic sound generators can be fixedly attached in the reactor housing 140 or can be movably attached in the reactor housing 140 so that fluid flow causes the acoustic sound generators to move or rattle. In discs 130A, 130B, the diameters of the apertures 131 and 132 may be the same or different and the orientations of the radial planes of the first and second discs 130A, 130B may be different. The present disclosure is not limited to including one or two acoustic sound generators, and may include any suitable number of acoustic sound generators that can sufficiently disturb the flow of fluid to create detectable sounds in the reactor 102.

In some aspects, the acoustic signal generator(s) used in the reactor 102 can be configured to generate sufficient sound that is detectable by microphone 114 only when a predetermined amount of fluid is flowing through the reactor. For example, the acoustic signal generator may be configured to generate a sound that is sufficiently loud to be detected by microphone 114 when the flow rate through the reactor 102 is greater than 1 gpm, greater than 3 gpm, or greater than 5 gpm, e.g., within a range of from 1 to 8 gpm. The present disclosure is not limited to these flow rates and the acoustic signal generator may be configured to generate the sound at any suitable flow rate that requires an increase in the amount of UV light generated. Alternatively, the controller 150 can be programmed to identify a threshold signal value from the detected sound that corresponds to a minimum flow rate.

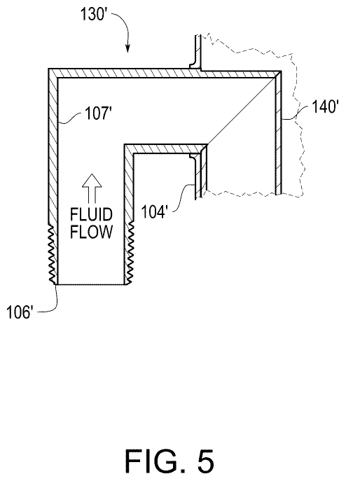

FIG. 5 is a partial cross-sectional view of the reactor housing 140′ in another embodiment which shows a bent inlet conduit 107′ that acts as an acoustic signal generator 130′. In this regard, the inlet conduit 107′ can be formed such that the fluid flow changes directions shortly after entering the inlet 106′. For example, the inlet conduit 107′ may have a portion that is elbow shaped, such that water traveling through the inlet 106′ quickly changes direction by about 180° around the outer reactor wall 104′. In this regard, the abrupt change in direction may occur within a length of the inlet conduit 107′ that is less than a length corresponding to five diameters of the inlet conduit 107′, such as from two to four diameter equivalent length. The change in direction of the flow causes turbulent regions that generate vibrations, which in turn generate the sound detected by the microphone 114. The present disclosure is not limited to changing the direction of the fluid flow by 180° and may be configured to change the direction of the fluid flow by any amount, such as from 45° or 220°, or 120° to 200°, that can cause sufficient sound in the reactor housing 140′ that can be detected by the microphone 114.

In some embodiments, the reactor 102 does not include a separate acoustic signal generator that is added to specifically generate sound from the fluid flow, and the microphone 114 can instead be configured to detect flow in the reactor based on sounds caused by the fluid flowing normally in the reactor, as discussed in more detail below.

Referring to FIG. 4, the reactor 102 of treatment system 100 includes the light source assembly 120 that may be removably coupled to the reactor 102. The light source assembly 120 includes at least one light source unit 122 that is arranged inside the light source assembly 120 that can emit UV radiation inside the treatment chamber 110 to treat the fluid flowing through the chamber 110 with the UV radiation for disinfection, purification, sterilization, or the like. The light source assembly 120 can be a UV lamp with light source unit(s) 122 that are filaments, or instead may include light source unit(s) that are UV LEDs. As shown in FIGS. 2 and 4, the light source assembly 120 may be a rod that is arranged in the treatment chamber 110 along the longitudinal axis L and configured to emit UV radiation radially outward within the treatment chamber 110. However, the present disclosure is not limited to this arrangement, and the light source assembly 120 may be arranged in any suitable manner. In this regard, the light source assembly 120 could be arranged on an inner wall of treatment chamber 110 and configured to emit UV radiation radially inwardly within the treatment chamber 110. The light source unit 122 could also be arranged at either or both ends of the treatment chamber and configured to emit UV radiation in a direction that is aligned with the longitudinal axis L. The light source assembly 120 can also be any shape including rod-shaped, disc-shaped, puck-shaped, etc.

The at least one light source unit 122 may emit light in the UV spectrum, for example, in a wavelength band of about 100 nm to about 405 nm, a wavelength band of about 140 to about 330 nm, or a wavelength band of about 180 nm to about 280 nm. The UV light in the above wavelength bands has high germicidal efficacy and may kill at least 99% of microorganisms, such as bacteria, fungi, viruses, mold, and the like, in the fluid, making the fluid safe for use and consumption. The at least one light source unit 122 may have an efficiency in converting electrical energy to UV light energy in a range of about 3% to about 30%, a range of about 4% to about 15%, or a range of about 5% to about 10%. The treatment system 100 may be designed to deliver a UV dose of 5 mJ/cm2 to 100 mJ/cm2, or about 30mJ/cm2, to the fluid at the target flow rate and target water quality, or may be designed to deliver any other suitable UV dose to the fluid. The at least one light source unit 122 may be, for example, an ultraviolet LED or other ultraviolet light producing lamp.

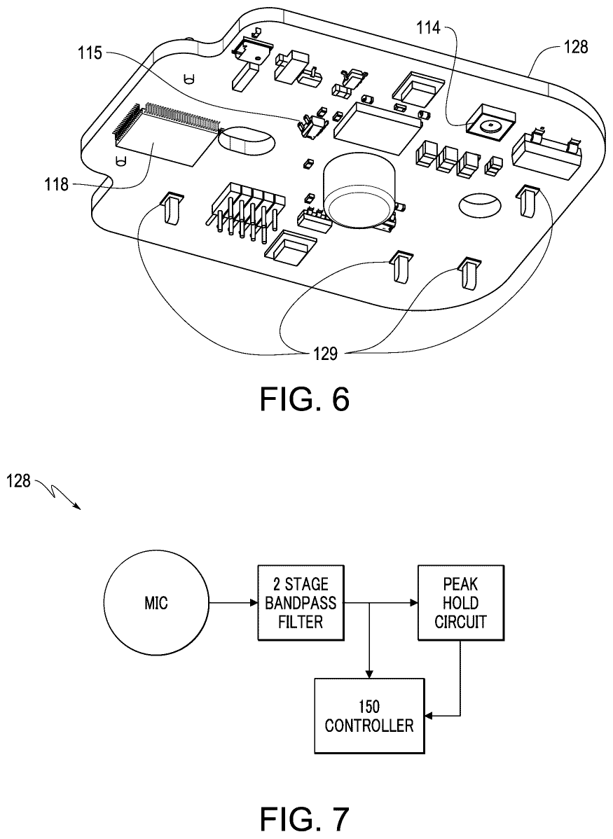

Referring to FIGS. 4 and 6, the reactor 102 of the treatment system 100 includes a circuit board 128, such as a printed circuit board (PCB) or a metal core printed circuit board (MCPCB), which is also mounted in the reactor 102. The circuit board 128 may be inset inside the reactor 102 and may be positioned at one end of the light source assembly 120. For example, the circuit board 128 may be positioned at the end of the light source assembly 120 that is closest to the inlet 106 or the acoustic signal generators 130A, 130B. A plane of the circuit board 128 may be oriented parallel to a width dimension of the light source assembly 120 and the chamber 110, and transverse to the longitudinal axis L.

The circuit board 128 is electrically connected to the controller 150 such that the circuit board 128 can send signal information to the controller 150 that the controller 150 uses to determine whether fluid is flowing through the reactor 102, the flow rate of the fluid, and/or that one or more components are defective. As shown in FIG. 6, the circuit board 128 includes the microphone 114, which is configured to detect the sound generated when fluid is flowing through the reactor 102. The microphone is configured to detect frequencies of 40 Hz-20,000 Hz, 75 Hz-5,000 Hz, or 100 Hz-2,000 Hz. However, the present disclosure is not limited to this range of frequencies and the microphone 114 may be configured to detect any range of frequencies such that the microphone 114 detects the sound generated by fluid flowing through the reactor 102, the sound generated by the acoustic signal generator 130, or another sound.

Referring to FIG. 4, in an embodiment, the microphone 114 detects the vibrations of the reactor housing 140 via coupler 116 positioned to directly contact the circuit board 128 at one of its ends, and directly contact a part of the reactor housing 140 (in this case part of the light source assembly 120) at its other end. The coupler 116 is configured to transmit vibrations from the reactor housing 140 to the circuit board 128. The coupler 116 can have a cylindrical shape, and can be made of rubber, silicone rubber, or the like, for example. In this arrangement, when fluid is flowing through the reactor, the fluid flow (or optionally fluid flow with the acoustic signal generator 130) will cause the reactor housing 140 to vibrate, and the coupler 116 transmits the vibrations from the reactor housing 140 to the circuit board 128 where they are detected as sound by the microphone 114. Directly coupling the microphone 114 to the reactor housing 140 in this way improves the ability of the microphone 114 to detect vibrations resulting from fluid flowing through the reactor 102.

The present disclosure is not limited to the arrangement of the microphone 114 on the circuit board 128. The microphone 114 may be arranged in any suitable location within the system 100 for detecting the sound generated by fluid flowing through the reactor 102. The light source assembly 120, the reactor 102, and/or the system 100 may include any other suitable sensor in place of or in addition to the microphone 114, and may include multiple microphones 114 that are similarly configured to send signals to the controller 150. In addition to microphones, any sound detection sensor (i.e., a vibration sensor) can be used that can detect sound (i.e., acoustic waves or mechanical vibrations), such as a piezoelectric accelerometer.

Referring to FIG. 6, the circuit board 128 may further include a connector, such as connector ports 129, for electrically connecting the circuit board 128 to connector pins extending from the controller 150. For example, the connector ports 129 of the circuit board 128 may be connected to one end of corresponding connector pins from the controller 150. The controller 150 may include four connector pins that extend from the at least one processor. However, the present disclosure is not limited to four pins and the processor may include any number of suitable pins for communicating with the microphone 114 and controlling the operations of the light source assembly 120.

Referring again to FIG. 1, the controller 150 is connected to a power source, such as an electrical grid, via the plug 151. The controller 150 can be configured to control the transmission of electrical power to the system 100 so as to control an intensity of light produced by the light source assembly 120. For instance, the controller 150 may transmit power from the electrical grid to the light source assembly 120, via one of the pins, for powering the at least one light source unit 122 and any sensors, such as the microphone 114 on the circuit board 128. The controller 150 may also control the transmission of electrical power to any other components and/or sensor arranged in the fluid treatment system 100.

The controller 150 may be configured to control the functioning of the reactor 102 based on measurements received from one or more sensors, including, for example, the microphone 114 and any other sensor, according to the methods described below. For instance, the microphone 114 may transmit a signal (e.g., a voltage signal) to the controller 150 based on the measured sound via the circuit board 128 through one of the pins. The controller 150 may then use the signal to determine whether fluid is flowing through the reactor 102 and/or determine the flow rate of the fluid, and modulate the amount of light produced by the light source assembly 120 based on the determination. Methods for controlling the reactor 102 are described further below.

The controller 150 can include hardware, such as at least one processor (e.g., CPU), circuitry for processing digital signals, and/or circuitry for processing analog signals, for example. The controller 150 may include one or a plurality of processors, circuit devices (e.g., an IC), and/or circuit elements (e.g., a resistor, a capacitor) on a circuit board, for example. The controller 150 may be or form part of a specialized or general purpose computer or processing system. One or more controllers, processors, or processing units, memory, and a bus that operatively couples various components, including the memory to the processor, may be used. The controller 150 can include a module that performs the methods described herein. The module may be programmed into the at least one processor, or loaded from memory, storage device, or network or combinations thereof. The controller 150 may execute operating and other system instructions, along with software algorithms, machine learning algorithms, computer-executable instructions, and processing functions of the fluid treatment system.

The controller 150 may be operational with numerous other general purpose or special purpose computing system environments or configurations. Examples of well-known computing systems, environments, and/or configurations that may be suitable for use with the disclosed embodiments may include, but are not limited to, personal computer systems, server computer systems, thin clients, thick clients, handheld devices, such as tablets and mobile devices, laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

The present disclosure further relates to a non-transitory computer-readable storage medium configured to store a computer-executable program that causes a computer to perform functions, such as those for implementing the methods disclosed herein. The computer-readable storage medium may further store the real time data collected by the controller 150 and computer-executable instructions. The storage medium may include a memory and/or any other storage device. The memory may be, for example, random-access memory (RAM) of a computer. The memory may be a semiconductor memory such as an SRAM and a DRAM. The storage device may be, for example, a register, a magnetic storage device such as a hard disk device, an optical storage device such as an optical disk device, an internal or external hard drive, a server, a solid-state storage device, CD-ROM, DVD, other optical or magnetic disk storage, or other storage devices.

### Methods of Operating the Fluid Treatment System

Disclosed herein are methods of operating a system 100, such as but not limited to any of the above described systems, for treating a fluid that flows through the reactor 102 and is exposed to UV light emitted from the UV light source assembly 120.

### Detecting Flow and/or Flow Rate

One method includes detecting, with the microphone 114, that fluid is flowing through the system 100, and controlling, with the controller 150, electric power supplied to the UV light source assembly 120. For example, the controller 150 may receive signal information from the microphone 114, via one of the pins, to detect and measure sound and the microphone 114 may transmit the signal information to the controller 150 via the circuit board 128. The controller may be configured to detect the signal from the microphone 114 continuously, periodically, or intermittently and/or the controller 150 may detect the sound due to the occurrence of a trigger. The measured sound is delivered to the controller 150 via the circuit board 128, which outputs a signal to the controller 150 including a voltage value. This voltage value is used by the controller 150 to control the amount of electric energy supplied to the light source assembly 120 via two of the pins. In other implementations, the circuit board 128 may include an onboard processor 118 (e.g., a microprocessor) that analyzes the signal from the microphone 114 and outputs a determination or decision to the controller 150, rather than a voltage value. In either case, the information received by the controller 150 from the circuit board 128 is used to control the amount of electric energy supplied to the light source assembly 120, as discussed in detail below.

In some embodiments, the sound the microphone 114 detects is the sound generated by the acoustic signal generator 130 when fluid is flowing through the inlet conduit 107. Fluid flow through the acoustic signal generator 130 can generate sound that is proportional to the flow rate of the fluid such that the higher the flow rate, the greater the amount of sound generated. When a higher flow travels through the reactor 102, the microphone 114 detects a greater sound and causes the circuit board 128 to output a signal with a higher voltage value. The controller 150 then uses the higher voltage value to determine that fluid is flowing through the reactor 102. Details regarding how the processor uses the voltage value are discussed in more detail below.

The present disclosure is not limited to detecting sound generated by the acoustic signal generator 130 and the microphone 114 may be configured to detect any sound that indicates that fluid is flowing through the reactor 102. For example, the microphone 114 may be configured to detect the sound of fluid traveling through the reactor 102 without the acoustic signal generator 130. This sound is generated by incidents of turbulence, cavitation, fluctuations in pressure, and vibration of the reactor 102 as the fluid travels through the fluid treatment system 100.

Another example is that the microphone 114 may be configured to detect the sound of the operations of another system. For example, the microphone 114 may be configured to detect the sound made by a water heater, faucet, or valve operating, which each indicate that fluid is likely flowing through the system 100. As such, the controller 150 is able to rely on the operations of the other systems, serving as the acoustic signal generator 130, to control the amount of energy power being delivered to the light source assembly 120 so as to control the amount of light generated by the light source assembly 120. Alternatively or in addition, the microphone 114 may detect the sound resulting from the operation of these systems, such as the sound of the fluid contacting a surface after exiting a household faucet. The present disclosure is not limited to these examples and the microphone may be configured to detect any manner of sounds indicating that water is flowing through the reactor 102.

The controller 150 uses the output signal from the circuit board 128 to control the electric power supplied to the UV light source assembly 120 so that (i) an amount of UV light is adjusted based on the output signal in a first state in which the measured sound is above a threshold, and (ii) a lower average amount of UV light is produced by the UV light source assembly 120 in a second state in which the measured sound is below the threshold. The controller 150 may be configured to continuously, periodically, or intermittently compare the output voltage to the threshold to determine whether to switch to the first state or the second state. The controller 150 may be configured to compare the output voltage to the threshold for a predetermined amount of time before determining whether to switch to the first state or the second state.

The threshold may be the lowest detectable amount of sound (e.g., a detection threshold) of the microphone 114 or may be any other amount of sound indicative of fluid flowing through the reactor 102. The threshold may be a detected amount of voltage output from the circuit board 128. The circuit board 128 may receive the sound signal from the microphone 114 and process the signal before sending it to the controller 150. For example, the circuit board may filter the sound signal and store peak amplitude values of the sound signal. These stored amplitude signals are then output to the controller 150 and compared against the threshold.

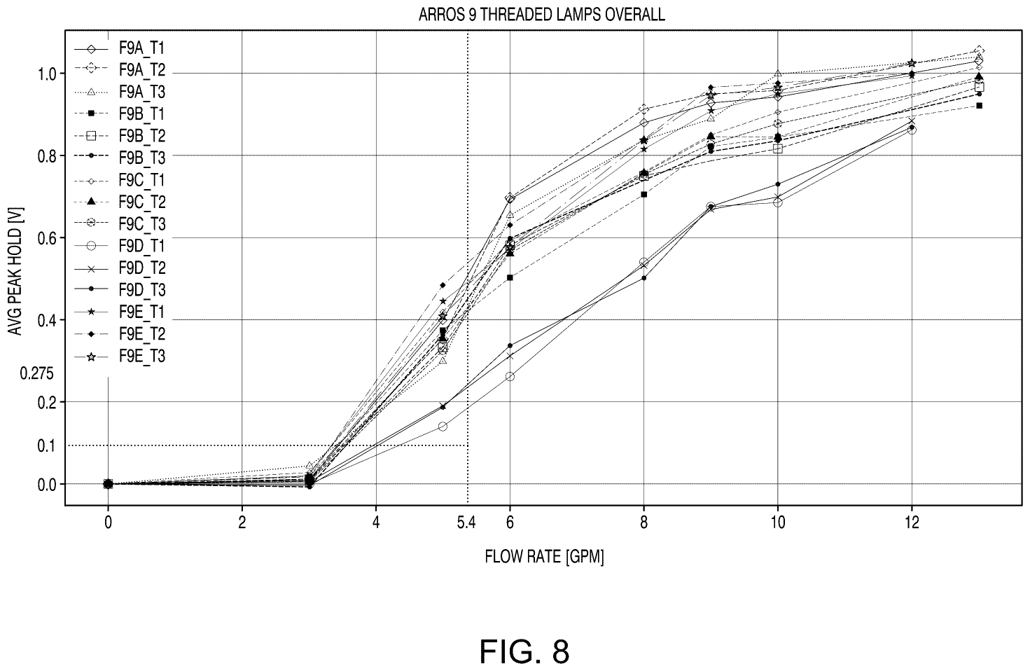

FIG. 8 is a graph showing the results of an experiment on treatment systems configured as shown in FIG. 4 in which the flow rate was controlled, sound was detected, and the average peak hold voltage was measured. The experiment included reading the average peak hold voltage while running flow through the treatment system 100 at 0 GPM, 3 GPM, 5 GPM, 6 GPM, 8 GPM, 9 GPM, 10 GPM, and 13 GPM three times for a single treatment system 100, shown in FIG. 8 as T1-T3. This process was performed for five sample treatment systems, shown as F9A-F9E in FIG. 8. Based on this experiment, an amount of voltage sent to the controller 150 by the circuit board 128 can be associated with the amount of flow through the reactor 102. For example, as indicated in FIG. 8, the threshold may be set to 0.1 V. Thus, if the circuit board 128 outputs a voltage greater than 0.1 V, this indicates to the controller 150 that fluid is flowing through the reactor 102 and that more electric power should be supplied to the UV light source assembly 120 so as to increase the amount of light produced. Of course, if the output voltage is less than 0.1 V, this indicates that fluid is not flowing through the reactor 102 or that fluid is flowing very slowly, and the processor will supply less electric power to the UV light source assembly 120 so as to reduce the amount of light produced. The present disclosure is not limited to a threshold voltage of 0.1 V and may be any suitable threshold for detecting that fluid is flowing through the reactor 102. The threshold is also not limited to a specific voltage and may be a particular pattern or waveform output by the circuit board 128 and received by the controller 150.

The first state, in which the output voltage indicates a flow rate greater than or equal to the threshold flow rate, may be the bulk of the flow rate range. For example, in the first state, the flow rate may be in a range of 2 to 25 gpm, 5 to 20 gpm, or 10 to 15 gpm, or any other suitable range depending on, for example, the particular system in which the reactor 102 is arranged. In the first state, the controller 150 may modulate the electric power supplied to the light source assembly 120 or light source assemblies if there is more than one based on the detected flow rate. For example, the controller 150 may adjust the amount of electric power supplied to the light source assembly 120 proportionally to the voltage output. If the measured sound increases, the controller 150 may increase the electric power supplied to the light source assembly 120 proportionally, and if the measured sound decreases, the controller 150 may decrease the electric power supplied to the light source assembly 120 proportionally. The electric power supplied to the light source assembly 120 may be adjusted in a stepwise or continuous manner proportionally to the output voltage or the electric power may be adjusted in any other suitable manner based on the output voltage. For example, the controller 150 may supply 100% power to the light source assembly 120 when the measured sound causes a voltage that is above the threshold and 60% when below the threshold.

In the second state, the voltage is less than the threshold, which indicates that the fluid in the reactor 102 is substantially stagnant. Instead of turning off power to the light source assembly 120 in the second state, the light source assembly 120 may be operated in a low flow or idle mode in which a low average amount of electric power is supplied to the light source assembly 120. This may conserve power while continuing to ensure adequate disinfection in the second state in which the fluid is substantially stagnant, and thereby improve efficiency of the system 100 and extend the lifetime of the at least one light source unit 122. In particular, when the controller 150 receives a voltage signal from the circuit board 128 that is below the threshold, the controller 150 may be configured to reduce power to the light source assembly 120, for example, to the at least one light source unit 122. For example, the controller 150 may switch to a low flow or idle mode in which a low average amount of electric power is supplied to the light source assembly 120. The low average amount of electric power has a non-zero value and is lower than the amount of electric power supplied to the light source assembly 120 in the first state over a given time. For example, to reduce the amount of energy consumed by the light source assembly 120, the controller 150 may control the electric power so that the low average amount of electric power supplied to the light source assembly 120 in a range of 40%-80%, 50%-70%, or 55%-65% of a maximum amount of electric power that can be supplied to the UV light source assembly 120. Of course, the present disclosure is not limited to these ranges and may be any suitable range of electric power suitable for ensuring that the substantially stagnant water is properly treated.

Unlike the first state, the low average amount of power supplied to the light source assembly 120 in the second state is not based on or adjusted based on the voltage output. Thus, neither the average amount of power nor the amount of power supplied to the light source assembly 120 in the second state is proportional to the detected flow rate. Nor is the amount of electric power supplied to the light source assembly 120 in the second state adjusted, in a proportional or other manner, based on changes in the flow rate in the second state. Instead, when the controller 150 determines that the detected flow rate is below the threshold flow rate, the controller 150 may determine that the system is in the second state, in which the fluid is substantially stagnant, and the controller 150 may switch to a low flow or idle mode in which a low average amount of electric power is supplied to the light source assembly 120 in the second state. The controller 150 may continue to supply the low average amount of electric power to the light source assembly 120 in the second state until or if the controller 150 determines that the detected flow rate is greater than or equal to the threshold voltage. Upon determining that the flow rate is greater than or equal to the threshold voltage, the controller 150 may determine that the system is in the first state, and may then switch to modulating the electric power based on the detected flow.

Low or no flow through the reactor 102 may mean, for example, that the fluid in the reactor 102 is substantially stagnant and may be in a steady state. In the substantially stagnant state and/or steady state, the fluid may have a flow rate of less than 2 gpm, 1 gpm or less, 0.5 gpm or less, or 0.25 gpm or less, including a flow rate of 0 gpm. As such, for example, the threshold amount of sound may be set to detect a threshold flow of greater than about 2 gpm, about 1 gpm, about 0.5 gpm, or about 0.25 gpm, or any other suitable value indicative of fluid flow above which higher UV power may be used.

By this method, power to the at least one light source unit 122 in the light source assembly 120 may be conserved during periods of low or no flow (e.g., when the fluid is substantially stagnant). This may extend the lifetime of the light source units and/or light source assembly 120.

Although embodiments disclosed herein have been described with respect to treating water and/or aqueous fluids with UV radiation treatment, the present disclosure is not limited to water and aqueous fluids, and may be used to treat any fluid, including liquids, vapors, gels, plasmas, and gases. Similarly, the present disclosure is not limited to residential UV treatment systems, and may be applied to industrial, municipal, and commercial systems.

### Filtering Signals From the Microphone

The circuit board 128 may be configured to filter out a range of frequencies which are not associated with the sound generated by the acoustic signal generator 130 when fluid is flowing through the inlet conduit 107. For example, the circuit board 128 may be configured to filter out signals outside of the range of 100 Hz to 5,000 Hz. The present disclosure is not limited to this range and may be configured to filter out any range of signals which are not associated with the sound generated when fluid is flowing through the reactor 102. The details of filtering the signal are discussed as follows.

Referring to FIG. 7, the circuit board 128 receives a waveform signal from the microphone 114 and filters the signal by attenuating undesired frequencies and/or amplifying desired frequencies. The circuit board 128 may then further filter the signal to output a narrower frequency band. In other words, the circuit board 128 may operate as a two-stage bandpass filter. After filtering the signal received from the microphone 114, the circuit board 128 may send the output signal to the controller 150, the output signal including the voltage value which is based on the filtered output signal. Of course, the signal filtering of the circuit board 128 is not limited to a two-stage bandpass filter and may include any suitable means of filtering the signal received from the microphone 114, including fewer or more stages of filtering.

After filtering the signal from the microphone 114, the circuit board 128 may store peak amplitude values of the signal from the microphone such that the circuit board 128 generates a waveform that represents maximum amplitudes of the bandpass filter output over time. In other words, the circuit board 128 may include a peak hold circuit that samples the filtered signal output from the circuit board 128 operating as a two-stage bandpass filter such that the circuit board 128 outputs a signal with a voltage value that corresponds to the maximum amplitude of the waveform received by the microphone 114. The circuit board 128 may also amplify the signal received from the microphone 114 before outputting the signal to the controller 150. The circuit board 128 includes an amplifier 115 and may be configured to amplify the signal by a specified gain. The sampling of circuit board 128, operating as a peak hold circuit, may use any suitable range of hold time and sampling rate such that the output signal may be used to detect that the output from the microphone 114 indicates that sound is being generated by fluid flowing through the reactor 102.

### Detecting Microphone Failure

Another method of operating the system 100 for treating a fluid that flows through the reactor 102 and is exposed to UV light emitted from the UV light source assembly 120 includes detecting, with at least the controller 150, that the microphone 114 is defective based on the measured voltage output from the circuit board 128. There is a problem that if the microphone 114 fails, the controller 150 may interpret receiving no signal, i.e., no sound detected, from the microphone 114 as indicating that fluid is not flowing through the reactor 102. Therefore, there is a risk that the system 100 may allow fluid to travel through the reactor while in the second state, in which a lower amount of electrical energy is delivered to the light source assembly 120. Accordingly there is a need to detect when the microphone 114 has failed.

Referring to FIG. 7, in an embodiment, after the circuit board 128 filters out a certain range of frequencies from the signal received from the microphone 114, the circuit board 128 may output the filtered signal directly to the controller 150, rather than through the peak hold circuit. The circuit board 128 can alternatively output the filtered signal to the peak hold circuit, and then to the controller 150. The controller 150 may then be configured to detect that the microphone 114 is defective if the microphone signal is oscillating or does not otherwise resemble a valid input. Also, a sensor (e.g., 4-20 mA) can provide a signal even when there is no flow and if the sensor is disconnected or damaged, the signal is lost.

In another embodiment, the system 100 may be configured to detect that the microphone 114 has failed by using an alarm. The system 100 may include an alarm, e.g., a speaker, which is used for alerting a user that a component within the system 100 has failed or that the system 100 requires the user to perform an operation. For example, the alarm may produce an intermittent “beep” that is audible to the user. The controller 150 may be configured to, at a predetermined interval, cause the alarm to make a sound in a frequency that the microphone 114 is able to detect. The controller 150 is electrically connected to the alarm such that the controller 150 can send a signal to the alarm causing the alarm to make a sound. If the microphone 114 is operational, the microphone 114 will detect the sound from the alarm and will transmit a signal to the controller 150 via the circuit board 128. Upon receiving the signal from the microphone 114, the controller 150 confirms that the microphone 114 is functioning properly. For example, the controller 150 may compare the signal from the microphone 114 to a predetermined value, the predetermined value being a value indicating that the signal received from the microphone 114 was generated based on the microphone 114 detecting the sound generated by the alarm.

Alternatively, if the microphone has failed, the microphone 114 will not detect the sound of the alarm and the controller 150 will not receive a sound signal from the microphone 114 or receives a sound signal that was not generated based on the microphone 114 detecting the sound generated by the alarm. In response to determining that the microphone 114 has failed, the controller 150 may be configured to respond by (i) changing to the first state, (ii) sending a signal to the alarm to generate a sound warning the user that the microphone 114 is not functioning, and/or both (i) and (ii).

It will be appreciated that the above-disclosed features and functions, or alternatives thereof, may be desirably combined into different systems and methods. Also, various alternatives, modifications, variations or improvements may be subsequently made by those skilled in the art, and are also intended to be encompassed by the disclosed embodiments. As such, various changes may be made without departing from the spirit and scope of this disclosure.

What is claimed is:

1. An apparatus for treating a fluid with ultraviolet (UV) radiation, the apparatus comprising:
a reactor through which the fluid flows in a flow path;
a UV light source that is located in the reactor and is configured to emit UV light into the fluid to treat the fluid as it flows through the reactor;
a vibration detection sensor that is located in the reactor and coupled to a housing of the reactor, the vibration detection sensor being configured to detect vibrations that vary based on a flow rate of the fluid through the reactor, and to output a signal based on the detected vibrations; and
a controller that is configured to receive the signal from the vibration detection sensor and control an intensity of the UV light based on the signal.

2. The apparatus of claim 1, wherein the apparatus further includes an acoustic signal generator that is located in the reactor and generates vibrations that vary based on a flow rate of the fluid.

3. The apparatus of claim 2, wherein flow through or over the acoustic signal generator produces vibrations with a distinct pattern or distinct waveform that is detectable by the vibration detection sensor.

4. The apparatus of claim 2, wherein the acoustic signal generator disturbs the flow of the fluid in the flow path and causes the fluid to flow more turbulently.

5. The apparatus of claim 2, wherein:
the reactor includes a conduit through which fluid flows through the reactor, and
the acoustic signal generator is a first disc that is located in the conduit, the first disc including a first aperture through which the fluid flows.

6. The apparatus of claim 5, wherein the conduit is an inlet conduit that receives fluid from an inlet of the reactor.

7. The apparatus of claim 5, further comprising a second acoustic signal generator that is a second disc that is located in the conduit spaced apart from the first disc in a direction of the fluid flow through the conduit, the second disc including a second aperture through which the fluid flows.

8. The apparatus of claim 7, wherein the first aperture and the second aperture each have a longest dimension in cross-section that is smaller than a longest dimension of the conduit in cross-section.

9. The apparatus of claim 2, wherein:
the acoustic signal generator includes a conduit section in the reactor that defines a part of the flow path and is bent so that the fluid flow changes direction in the conduit section in an amount in a range of from 45° to 220°.

10. The apparatus of claim 1, wherein the vibrations are generated by a device that is external to the apparatus for treating the fluid.

11. The apparatus of claim 1, wherein the controller controls the intensity of the UV light by controlling an amount of electrical power supplied to the UV light source.

12. The apparatus of claim 11, wherein the controller is configured to, in response to determining that a value of the signal is above a threshold amount, change an amount of electrical power supplied to the UV light from a first amount to a second amount, the second amount being larger than the first amount.

13. The apparatus of claim 11, wherein the controller is configured to, in response to determining that a value of the signal is below a threshold amount, decrease an amount of electrical power supplied to the UV light.

14. The apparatus of claim 1, further comprising a circuit board, wherein the circuit board is configured to receive the signal from the vibration detection sensor and process the signal before outputting the signal to the controller.

15. The apparatus of claim 14, wherein a value of the signal output by the circuit board is an amount of voltage corresponding to the vibrations detected by the vibration detection sensor.

16. The apparatus of claim 14, wherein the vibration detection sensor is coupled to the housing by a rubber coupler that is in contact with both the circuit board and the housing.

17. A method of operating the apparatus of claim 1, the method comprising:
causing a vibration generating device to generate a test vibration;
detecting, with the vibration detection sensor, the test vibration generated by the vibration generating device;
outputting a signal, with the vibration detection sensor, based on the test vibration detected from the vibration generating device; and
determining, with the controller, whether the vibration detection sensor is defective based on the outputted signal.

18. The method of claim 17, wherein the controller determines that the vibration detection sensor is defective when a value of the signal received from the vibration detection sensor does not match a predetermined value.

19. The method of claim 17, wherein, in response to determining that the vibration detection sensor is defective, changing, with the controller, an amount of electrical power supplied to the UV light from a first amount to a second amount, the second amount being larger than the first amount.

20. The apparatus of claim 1, wherein the vibration detection sensor includes a microphone or an accelerometer.
